(19) United States
(12) Patent Application Publication
(10) Pub. No.: US 2010/0010437 A1
(43) Pub. Date: Jan. 14, 2010

(54) STEERABLE CATHETER WITH DISTENDING LUMEN-ACTUATED CURLING CATHETER TIP

(76) Inventors: Robin R. Miles, Danville, CA (US); Daniel L. Schumann, Concord, CA (US); Mark A. Pearson, Pittsburg, CA (US); Dawn Hilken, Pleasant Hill, CA (US)

Correspondence Address: Lawrence Livermore National Security, LLC, LAWRENCE LIVERMORE NATIONAL LABORATORY, PO BOX 808, L-703, LIVERMORE, CA, 94551-0808, US

(21) Appl. No.: 12/502,097

(22) Filed: Jul. 13, 2009

Related U.S. Application Data
(60) Provisional application No. 61/080,164, filed on Jul. 11, 2008.

(51) Int. Cl. A61M 25/092

(52) U.S. Cl. 604/95.03

(57) ABSTRACT

A steerable catheter having a catheter tip connected to an elongated catheter body, with the catheter tip having an elastomeric cylindrical body and one or more steering lumens each offset from the longitudinal axis of the elastomeric cylindrical body and extending substantially the length of the elastomeric cylindrical body so that increasing the internal pressure of one or more of the steering lumens using, for example, a pneumatic or hydraulic pressure source or heating a thermally expandable media in the steering lumens, operates to curl the longitudinal axis of the elastomeric cylindrical body towards the internally pressurized steering lumen or lumens for steering the catheter tip.

### CROSS-REFERENCE TO RELATED APPLICATIONS

This application claims priority in provisional application no. 61/080164, filed on Jul. 11, 2008, entitled “Pneumatic/Hydraulic Articulating Catheter” by Robin R. Miles et al, incorporated by reference herein.

### FEDERALLY SPONSORED RESEARCH OR DEVELOPMENT

The United States Government has rights in this invention pursuant to Contract No. DE-AC52-07NA27344 between the United States Department of Energy and Lawrence Livermore National Security, LLC for the operation of Lawrence Livermore National Laboratory.

### FIELD OF THE INVENTION

The present invention relates to catheters, and more particularly to the steering and directional control of catheters using an elastomeric catheter tip that resiliently curls in response to and in the direction of one or more internally pressurized lumens which extend substantially the length of the elastomeric catheter tip from an offset position relative to the longitudinal axis.

### BACKGROUND OF THE INVENTION

Catheters, which generally consist of tubing through which dyes, balloons, and stents are deployed to a distal or leading end positioned in a patient's body, are increasingly used as an alternative to the more invasive open surgery in many medical procedures. Catheters are used, for example, to open clogged arteries in both the heart and brain, perform biopsies in cancer patients, short circuit the liver with a stent in patients with liver diseases, and inspect the body for a variety of reasons. Catheter procedures are generally shorter and safer than open surgery, and can be performed on an outpatient basis which can provide significant cost savings for medical establishments.

Despite their many benefits, the steering and maneuvering of catheters in a patient's body can be difficult since catheter control is generally performed from outside the body at a proximal end of the catheter opposite the leading or distal end, and it is often difficult to aim the distal end of the catheter in any particular direction to force the catheter down one capillary versus another, such as when a fork is encountered. As such, physicians using catheters to perform medical procedures on patients need to be very skilled to maneuver catheters into position.

### SUMMARY OF THE INVENTION

One aspect of the present invention includes a steerable catheter, comprising: an elongated catheter body having proximal and distal ends and a longitudinal axis; and a catheter tip having an elastomeric cylindrical body with a longitudinal axis and coaxially connected to the distal end of the elongated catheter body, and at least one steering lumen each offset from the longitudinal axis of the elastomeric cylindrical body and extending substantially the length thereof so that increasing the internal pressure of the steering lumen curls the longitudinal axis of the elastomeric cylindrical body towards the steering lumen for steering the catheter tip; and means for independently controlling the internal pressure of each steering lumen to control the direction and magnitude of the curl.

Another aspect of the present invention includes a steerable catheter tip, comprising: an elastomeric cylindrical body with a longitudinal axis and coaxially connectable to a distal end of an elongated catheter body, said elastomeric cylindrical body having at least one steering lumen each offset from the longitudinal axis of the elastomeric cylindrical body and extending substantially the length thereof so that increasing the internal pressure of the steering lumen curls the longitudinal axis of the elastomeric cylindrical body towards the steering lumen for steering the catheter tip

Generally, the present invention is directed to a steerable catheter having an elastomeric catheter tip connected to an elongated catheter body, with the catheter tip having an elastomeric cylindrical body and one or more steering lumens for curling, i.e. controlling the curvature of, the elastomeric catheter tip by controlling the internal pressures of the steering lumens. In this manner, the catheter tip may be curled in any direction to aid in directing the catheter up an appropriate capillary.

The basic approach is to extend one or more expandable steering lumens through substantially the length of the elastomeric catheter tip and offset from the longitudinal center axis of the elastomeric catheter tip, so that selectively expanding the steering lumen volumes operates to curl the catheter tip towards the expanded (distended) steering lumen or lumens. Expansion of the steering lumens may be effected at the proximal end either pneumatically (e.g. carbon dioxide) or hydraulically (e.g. saline solution) through an external source, or by phase change expansion of a thermally expandable media (e.g. solid to liquid wax). The amount of force generated in a small volume area using this technique of off-centered volume expansion in an elastomeric catheter tip can be quite high. By careful choice of the morphology of the expandable volume, useful work can be performed at the catheter tip to effect steering and direction control.

It is appreciated that the elongated catheter body, as used herein, includes both functional catheters i.e. having a tubular configuration surrounding a central lumen (such as 18 in FIG. 1), or guidewires which are known in the art whose sole purpose is to be directed to a particular location in the body of a patient, and once at that location to serve as a guide for inserting of a functional catheter.

And various types of thermally expandable media may include, for example, silicone which expands about 0.1%/° C., phase changing polymers which expand about 40% upon melting, or polymers with low boiling points to obtain actuation through gas formation. Actuation temperatures near body temperature can be chosen to minimize the possibility of hyperthermia of the cells. Another example uses laser light to heat the polymer similar to that used in synergistic projects such as those proposed to deploy devices to clear arterial blockages causing strokes.

The primary application of the directed movement/steering of a catheter provided by the present invention would be for medical catheters to facilitate deployment/positioning of the catheter to the desired internal region of a patient. However, catheter tip steering may also be useful in other non-medical applications, such as for example performing inspections of devices, machines, or systems in hard-to-reach areas.

### BRIEF DESCRIPTION OF THE DRAWINGS

The accompanying drawings, which are incorporated into and form a part of the disclosure, are as follows:

FIG. 1 is a schematic cross-sectional view of an exemplary embodiment of the steerable catheter of the present invention, shown having an elastomeric catheter tip connected to a semi-rigid elongated catheter body, and supplied with pressurized fluid from a pressurized fluid source.

FIG. 2 is an enlarged cross-sectional view of the exemplary elastomeric catheter tip taken along line 2-2 of FIG. 1, shown having two steering lumens surrounding a central lumen.

FIG. 3 is an enlarged cross-sectional view of the exemplary connector shown in FIG. 1.

FIG. 4 is a cross-sectional view of the elastomeric catheter tip of FIG. 1 after the internal pressure in steering lumen 17′ is increased and distended, and illustrating the curling of the elastomeric catheter tip for steering.

FIG. 5 is an enlarged cross-sectional view of the curled elastomeric catheter tip of FIG. 4, taken along line 5-5 of FIG. 4, and showing the enlarged diameter of distended steering lumen 17.

FIG. 6 is a schematic cross-sectional view of another exemplary embodiment of the steerable catheter of the present invention, shown having an elastomeric catheter tip connected to a semi-rigid elongated catheter body, and having a heat controller for supplying heat to the expandable media in the steering lumens.

FIG. 7 is an enlarged view cross-sectional view of another exemplary embodiment of the elastomeric catheter tip similar to FIG. 6, but with the steering lumens being self-contained and sealed to localize pressure.

FIG. 8 is a cross-sectional view of another exemplary embodiment of the elastomeric catheter tip, and similar to FIG. 2, showing a single steering lumen surrounding a central lumen formed by a tubular configuration.

FIG. 9 is a cross-sectional view of another exemplary embodiment of the elastomeric catheter tip, and similar to FIG. 2, showing four steering lumens surrounding a central lumen formed by a tubular configuration.

FIG. 10 is a cross-sectional view of another exemplary embodiment of the elastomeric catheter tip, and similar to FIG. 8, showing four steering lumens embedded in an elastomeric cylinder configuration.

### DETAILED DESCRIPTION

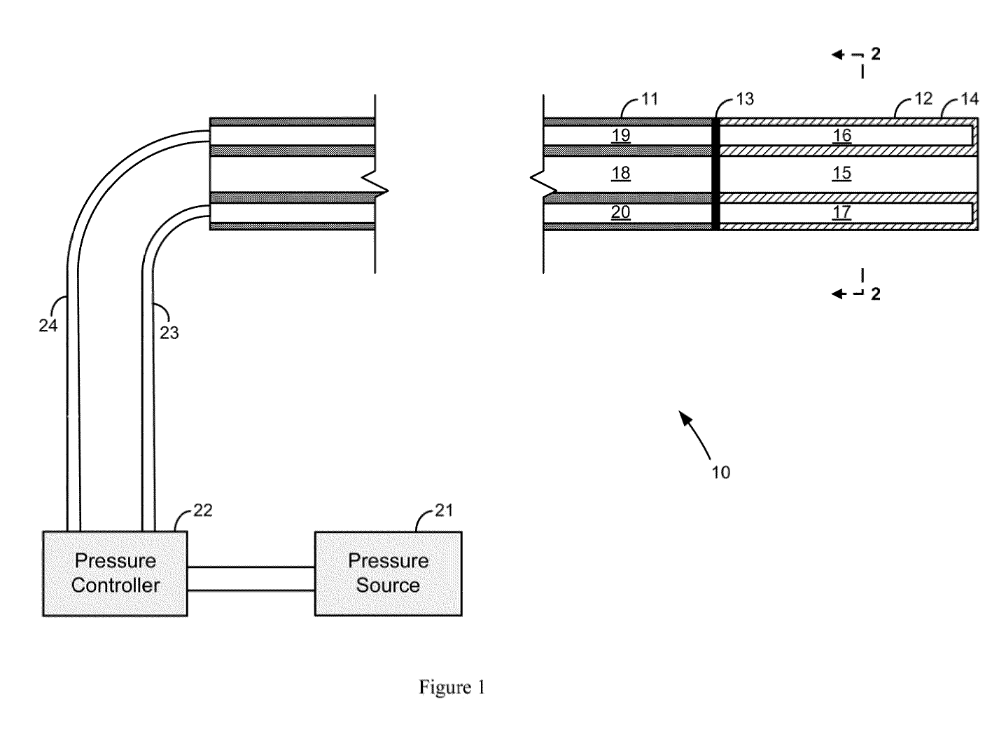

Turning now to the drawings, FIGS. 1-3 show a first exemplary embodiment of the steerable catheter of the present invention, and generally indicated at reference character 10. In particular, FIG. 1 is a schematic cross-sectional view of the steerable catheter, and shown having an elastomeric catheter tip 12 connected to a (preferably semi-rigid) elongated catheter body 11, and a pressurized fluid source 21 for selectively and independently supplying selected steering lumens in the elastomeric catheter tip 12 with pressurized fluid via the elongated catheter body 11.

As can be seen in FIGS. 1 and 2, the elastomeric catheter tip 12 has a generally cylindrical body that is shown in particular as an elastomeric tubular sidewall 14. In the tubular configuration, a central lumen is formed surrounded by the elastomeric tubular sidewall. In either case, the cylindrical body/tubular sidewall 14 has a longitudinal axis along which the length of the cylindrical body/tubular sidewall 14 and the elastomeric catheter tip 12 is defined. And extending through substantially the entire length of the cylindrical body 14 is at least one, and preferably two or more, steering lumens (e.g. 16 and 17). In particular, as shown in FIG. 2, the steering lumens are shown integrally formed in and as part of the tubular sidewall 14 which surrounds a central lumen 15. Each steering lumen 16 and 17 is shown radially offset from the central lumen 15 and the longitudinal axis (not shown) of the cylindrical body 14. The inner radial wall thickness between each of the steering lumens 16, 17 and the central lumen 15 is preferably greater than the outer radial wall thickness between each of the steering lumens 16, 17 and an outer surface of the tubular sidewall 14. While various elastomeric materials may be used for the cylindrical body of the catheter tip, one exemplary material is silicone.

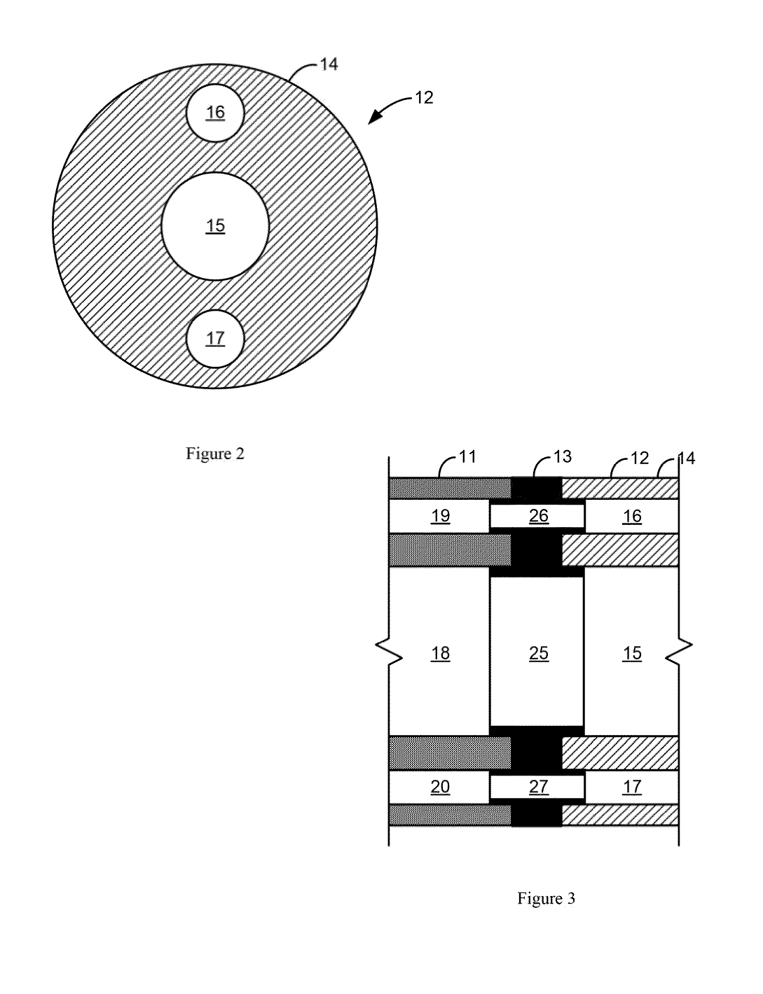

The elastomeric catheter tip 12 is connected to a distal end of the elongated catheter body 11 so that each of the fluid conduits, i.e. pressure lines 19, 20 extending through the elongated catheter body 11 are communicably aligned with and connected to a corresponding one of the steering lumens 16, 17. Furthermore, the central lumen 18 of the elongated catheter body 11 and the central lumen 15 of the elastomeric catheter tip are also connected and communicably aligned. Various methods of connecting the elastomeric catheter tip 12 to the distal end of the elongated catheter body 11 may be used. FIGS. 1 and 3 show one exemplary connector 13 for end/butt connecting the two components together. In particular, the connector 13 is shown having opposing nipples for connecting the steering and central lumens 15-17 to the pressure lines and central lumen 18-20, with the nipples having channels 25-27 therethrough which provide the fluidic communication between the catheter body and the catheter tip. It is appreciated that the elongated catheter body 11 may be constructed using a rigid or semi-rigid material, such as for example, polyether block amide (PEBA).

A pressure controller 22 is also shown in FIG. 1 for selectively and independently controlling the level of pressurized fluid delivered to each of the steering lumens (as represented by separate pressure lines 23, 24) from the pressurized fluid source. It is appreciated that the pressure controller 22 may include valves and valve control devices/electronics as known in the art. The pressure lines 23, 24 are shown connected to respective pressure lines (lumens) 20, 19 extending through the elongated catheter body 11 which function as the fluid conduit in the catheter body for channeling pressurized fluid from the pressurized fluid source. The pressure lines 19, 20 may, for example, be molded or extruded into the walls of the elongated catheter body 11. The pressure controller 22 operates to select the pressure line and corresponding steering lumen, and controls the pressure provided thereto from the pressure source 21.

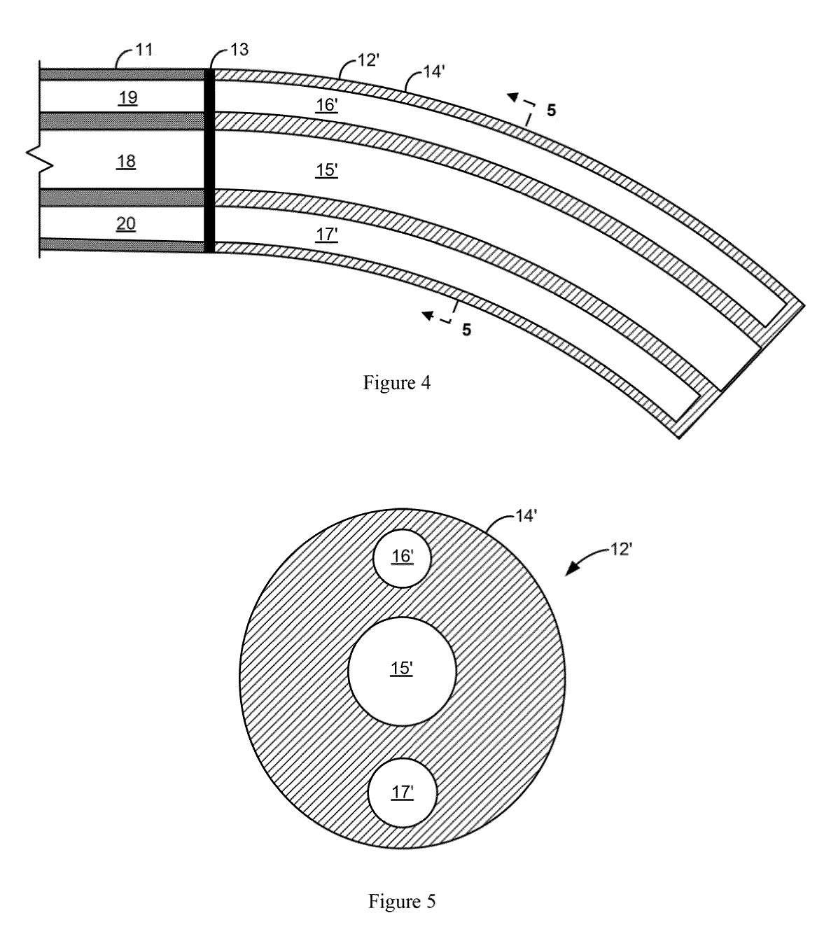

FIGS. 4 and 5 illustrate the steering that is achieved by curling of the elastomeric catheter tip 12′ of the present invention. In particular, FIG. 4 is a cross-sectional view of the elastomeric catheter tip of FIG. 1 after the internal pressure in steering lumen 17′ is increased and distended, and FIG. 5 is an enlarged cross-sectional view of the curled elastomeric catheter tip of FIG. 4, taken along line 5-5 of FIG. 4, and showing the enlarged diameter of distended steering lumen 17′. It is notable that the “curling” of the elastomeric catheter tip is not a jointed bending/hinging of segments at various joints/hinge points. Rather it produces a continuously bent curve along the entire length of the catheter tip. As previously discussed, the inner radial wall thickness between each of the steering lumens 16, 17 and the central lumen 15 is preferably greater than the outer radial wall thickness between each of the steering lumens 16,17 and an outer surface of the tubular sidewall 14. This is so that when the steering lumen is pressurized and distended, such as 17′ in FIGS. 4 and 5, there is greater relative expansion along the radially outer region of the tubular sidewall, than the radially inner region, in order to cause the longitudinal axis of the elastomeric catheter tip to curl towards and in the direction of the pressurized steering lumen. In any case, the magnitude and direction of the curling is controlled by selecting one or more of the steering lumens to the exclusion of others, and varying the pressure level directed into the selected steering lumen.

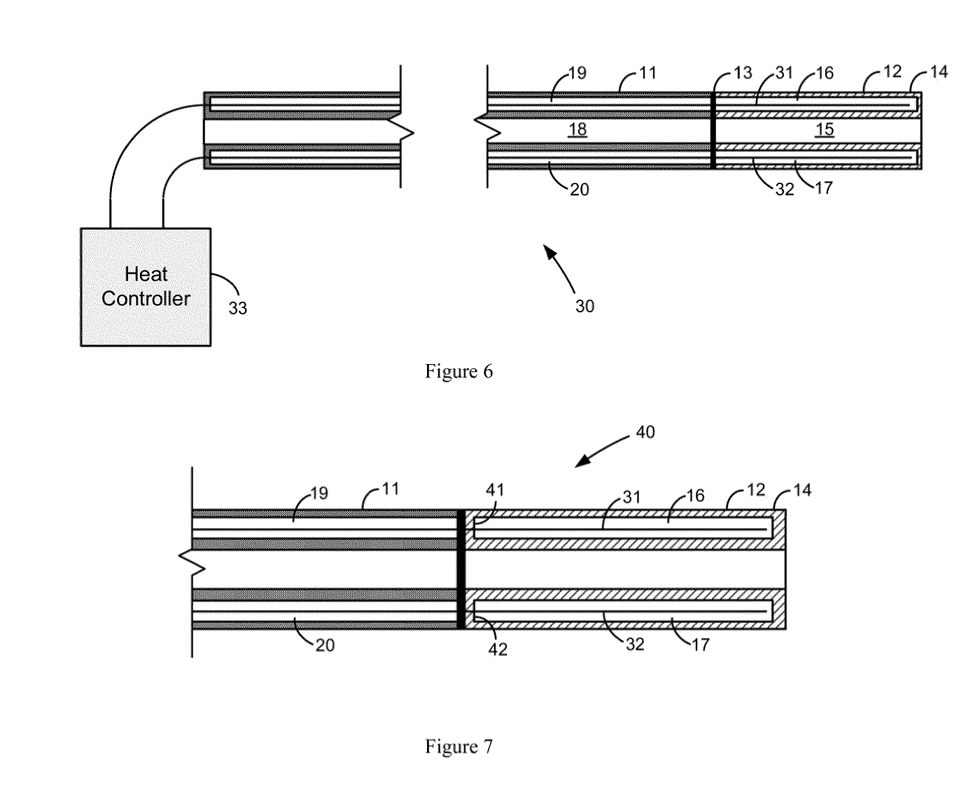

FIG. 6 is a schematic cross-sectional view of another exemplary embodiment of the steerable catheter of the present invention, generally indicated at 30. The steerable catheter is shown having an elastomeric catheter tip 12 connected to a semi-rigid elongated catheter body 11 in a manner similar to that previously discussed. However, here each of the steering lumens contains thermally expandable media, such as for example silicone, and a heat controller 33 is provided for independently heating the thermally expandable media in each of steering lumens. It is appreciated that the heat controller may include any variety of heating and control mechanisms (e.g. resistive, optical, etc.) as known in the art. In FIG. 6, heater wires 31 and 32 are shown passed into the steering lumens 16 and 17 from the heat controller 33 which selects one or more of the steering lumen to be pressurized.

FIG. 7 is an enlarged view cross-sectional view of another exemplary embodiment of the elastomeric catheter tip similar to FIG. 6 using heat controlled distending of the steering lumens. Unlike the open configuration of the steering lumens in FIGS. 1-3, the steering lumens 16 and 17 are shown sealed and self-contained by an additional wall, 14 for steering lumen 16, and 42 for steering lumen 17. In this manner, only a small local volume needs to be heated and pressurized for potentially faster response times.

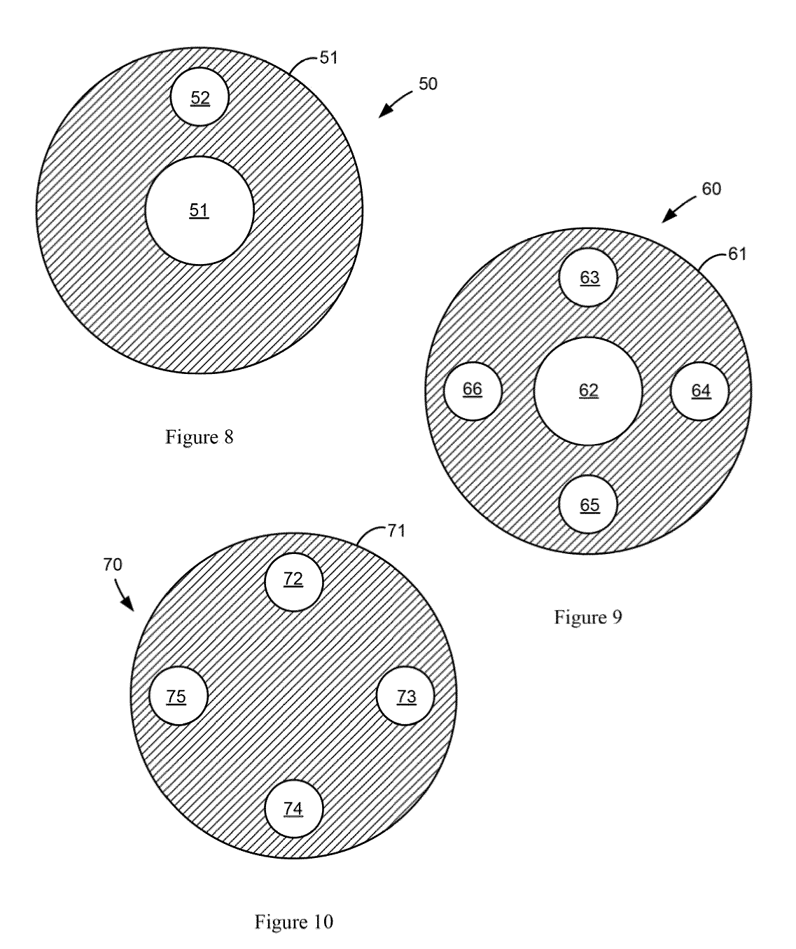

FIGS. 8-10 show additional exemplary embodiments of the elastomeric catheter tip of the present invention. In particular, FIG. 8 is a cross-sectional view similar to FIG. 2 of an elastomeric catheter tip 50 showing a single steering lumen 52 surrounding a central lumen which is formed by a tubular sidewall configuration 51. Such a single steering lumen embodiment would need to be rotated about its longitudinal axis in order to change directions of the elastomeric catheter tip. FIG. 9 is a cross-sectional view an elastomeric catheter tip 50 which is similar to FIG. 2, but having four steering lumens 63-66 surrounding a central lumen 62. Here too, the cylindrical body of the catheter tip is a tubular sidewall 61 which forms the central lumen 62. And FIG. 10 is a cross-sectional view of an elastomeric catheter tip 70 that is not tubular. Four steering lumens 72-74 are shown radially offset from a central axis (not shown) and embedded in the elastomeric cylinder body 71.

While particular operational sequences, materials, temperatures, parameters, and particular embodiments have been described and or illustrated, such are not intended to be limiting. Modifications and changes may become apparent to those skilled in the art, and it is intended that the invention be limited only by the scope of the appended claims.

We claim:

1. A steerable catheter, comprising:
an elongated catheter body having proximal and distal ends and a longitudinal axis; and
a catheter tip having an elastomeric cylindrical body with a longitudinal axis and coaxially connected to the distal end of the elongated catheter body, and at least one steering lumen each offset from the longitudinal axis of the elastomeric cylindrical body and extending substantially the length thereof so that increasing the internal pressure of the steering lumen curls the longitudinal axis of the elastomeric cylindrical body towards the steering lumen for steering the catheter tip; and
means for independently controlling the internal pressure of each steering lumen to control the direction and magnitude of the curl.

2. The steerable catheter of claim 1,
wherein said means for independently controlling the internal pressure of each steering lumen includes fluid conduit means extending through the elongated catheter body for channeling pressurized fluid from a pressurized fluid source to each of the steering lumens.

3. The steerable catheter of claim 2,
further comprising the pressurized fluid source which is selected from a group consisting of a pneumatic pressure source and a hydraulic pressure source.

4. The steerable catheter of claim 1,
wherein each of the steering lumens contains thermally expandable media, and said means for independently controlling the internal pressure of each steering lumen includes means for independently heating the thermally expandable media in each steering lumen.

5. The steerable catheter of claim 1,
wherein the elastomeric cylindrical body is an elastomeric tubular sidewall surrounding a central lumen, with the steering lumens formed in the elastomeric tubular sidewall.

6. The steerable catheter of claim 5,
wherein for each steering lumen, an inner radial wall thickness between the steering lumen and the central lumen is greater than an outer radial wall thickness between the steering lumen and an outer surface of the elastomeric tubular sidewall.

7. A steerable catheter tip, comprising:
an elastomeric cylindrical body with a longitudinal axis and coaxially connectable to a distal end of an elongated catheter body, said elastomeric cylindrical body having at least one steering lumen each offset from the longitudinal axis of the elastomeric cylindrical body and extending substantially the length thereof so that increasing the internal pressure of the steering lumen curls the longitudinal axis of the elastomeric cylindrical body towards the steering lumen for steering the catheter tip.

8. The steerable catheter tip of claim 7,
wherein the elastomeric cylindrical body is an elastomeric tubular sidewall surrounding a central lumen, with the steering lumens formed in the elastomeric tubular sidewall.

9. The steerable catheter tip of claim 8,
wherein for each steering lumen, an inner radial wall thickness between the steering lumen and the central lumen is greater than an outer radial wall thickness between the steering lumen and an outer surface of the elastomeric tubular sidewall.
